(19) United States
(12) Patent Application Publication
(10) Pub. No.: US 2024/0212105 A1
(43) Pub. Date: Jun. 27, 2024

(54) SYSTEMS AND METHODS FOR AMBIENT LIGHT COMPENSATION IN MEDICAL IMAGING

(71) Applicant: Stryker Corporation, Kalamazoo, MI (US)

(72) Inventor: Paul Roald WESTWICK, Vancouver (CA)

(73) Assignee: Stryker Corporation, Kalamazoo, MI (US)

(21) Appl. No.: 18/540,822

(22) Filed: Dec. 14, 2023

Related U.S. Application Data
(60) Provisional application No. 63/476,639, filed on Dec. 21, 2022.

(51) Int. Cl. G06T 5/50; G06T 3/40

(52) U.S. Cl. CPC G06T 5/50; G06T 3/40; G06T 2207/20224

(57) ABSTRACT

A method for compensating for ambient light in medical imaging includes receiving an ambient light image and a fluorescence image; determining a level of ambient light in the ambient light image; and in accordance with determining that the level of ambient light in the ambient light image meets a threshold, generating an ambient light compensated image based on the ambient light image and the fluorescence image that compensates for contributions of the ambient light to the fluorescence image.

### CROSS-REFERENCE TO RELATED APPLICATIONS

This application claims the benefit of U.S. Provisional Application No. 63/476,639, filed Dec. 21, 2022, the entire contents of which are hereby incorporated by reference herein.

### FIELD

This disclosure relates generally to the field of medical imaging. More specifically, this disclosure relates to ambient light compensation in medical imaging.

### BACKGROUND

Medical imaging systems, such as open field medical imaging systems and endoscopic imaging systems for minimally-invasive surgery, can provide clinical information for medical practitioners who need to make decisions (for example, intraoperative or treatment decisions) based on visualization of tissue. One area of particular growth in medical imaging systems involves fluorescence imaging. Medical imaging systems capable of fluorescence imaging excite endogenous or exogenously introduced fluorophores and image the fluorescence emitted by the fluorophores.

The intensity of the fluorescence light emitted by the fluorophores during fluorescence imaging is relatively low. Therefore, fluorescence imaging can be susceptible to light pollution resulting from light having a bandwidth that overlaps with the fluorescence light interfering with detection of the fluorescence light. For example, ambient light from room lights may interfere with the detection of fluorescence light by open-field imaging systems.

Imaging systems can be configured to compensate for ambient light by capturing ambient light frames in alternating fashion with fluorescence light frames. A given ambient light frame may be used to subtract out the ambient light contribution to a fluorescence light frame captured close in time to the ambient light frame. Although this method can be effective at compensating for the effect of ambient light in the fluorescence light frames, it may produce noisier fluorescence light frames, and, as such, may not be desirable in all situations. Further, because the fluorescence light frames and the ambient light frames are captured at different times, motion of the target may result in the brightness at a given pixel location in the fluorescence light frame no longer corresponding to the same location in the ambient light frame, resulting in phantom fluorescence artifacts once ambient light compensation is performed.

### SUMMARY

According to one aspect, systems and methods can include selectively performing ambient light compensation in fluorescence imaging only when the intensity of the ambient light is sufficiently high. A level of ambient light intensity of an ambient light image may be compared to a threshold. Ambient light compensation in a fluorescence image may be performed using the ambient light image only if the level of ambient light intensity is sufficiently high to meet the threshold. The amount of ambient light compensation may be scaled based on the degree to which the level of ambient light exceeds the threshold. An upper threshold may be used such that full ambient light compensation is performed when the level of ambient light is above the upper threshold.

According to another aspect, systems and methods can include using motion compensation techniques when combining images corresponding to different light sources, such as for ambient light compensation for fluorescence imaging, to reduce motion-related artifacts resulting from the different light source images being captured at different times. An imaging system captures a time series of images that includes images corresponding to different light sources (e.g., fluorescence light emission from fluorophores and ambient light), including a first light source series corresponding to a first light source and a second light source series corresponding to a second light source, with images of the first light source series being captured in alternating fashion with images of the second light source series. The times series of images may be limited to just these two series or may include one or more other light source image series as well. At least one series of images of the time series of images is used to determine an estimate of optical flow between two images in the series. The estimate of optical flow is used to generate an estimate of an image of the first light source series captured at the capture time of an image from the second light source image. The estimated first light source series image can then be combined with the second light source series image with reduced motion-based artifacts.

According to an aspect, a method for compensating for ambient light in medical imaging includes: receiving an ambient light image and a fluorescence image; determining a level of ambient light in the ambient light image; and in accordance with determining that the level of ambient light in the ambient light image meets a threshold, generating an ambient light compensated image based on the ambient light image and the fluorescence image that compensates for contributions of the ambient light to the fluorescence image.

Determining the level of ambient light in the ambient light image may include determining a proportion of the ambient light image that has pixel intensity values that are above a predetermined amount.

The threshold may correspond to a proportion of an image having pixel values above a predetermined amount.

The ambient light compensated image may include the fluorescence image modified based on the ambient light image.

The ambient light compensated image may include a combination of a reflected light image and the fluorescence image modified to compensate for the contributions of the ambient light to the fluorescence image. Generating the ambient light compensated image may include generating a compensated fluorescence image by subtracting at least a portion of the ambient light image from the fluorescence image; and combining the compensated fluorescence image with the reflected light image.

Generating the compensated fluorescence image may include scaling pixel values of at least one of the ambient light image and the fluorescence image based on at least one of a difference in exposure period and a difference in gain.

Combining the compensated fluorescence image with the reflected light image may include scaling pixel values of at least one of the compensated fluorescence image and the reflected light image based on at least one of a difference in exposure period and a difference in gain.

The method may include, in accordance with determining that the level of ambient light in the ambient light image does not meet the threshold, displaying the fluorescence image or an image generated based on the fluorescence image without compensating for the ambient light.

Generating the ambient light compensated image may include scaling pixel values of the ambient light image by a scaling factor that corresponds to an amount that the level of ambient light in the ambient light image is above the threshold.

The threshold may be a lower threshold, and generating the ambient light compensated image may include: scaling pixel values of the ambient light image by a scaling factor when the level of ambient light in the ambient light image is above the lower threshold and below an upper threshold; and not scaling the pixel values of the ambient light image by the scaling factor when the level of ambient light in the ambient light image is above the upper threshold.

The ambient light compensated image may be generated based on an estimated ambient light image that is an estimate of the ambient light at a capture time of the fluorescence image.

The method may include displaying the ambient light compensated image. The method may include generating and displaying a visual guidance based on the ambient light compensated image.

According to an aspect, a system includes one or more processors, memory, and one or more programs stored in the memory for execution by the one or more programs, the one or more programs including instructions for: receiving an ambient light image and a fluorescence image; determining a level of ambient light in the ambient light image; in accordance with determining that the level of ambient light in the ambient light image meets a threshold, generating an ambient light compensated image based on the ambient light image and the fluorescence image that compensates for contributions of the ambient light to the fluorescence image; and displaying the ambient light compensated image.

Determining the level of ambient light in the ambient light image may include determining a proportion of the ambient light image that has pixel intensity values that are above a predetermined amount.

The threshold may correspond to a proportion of an image having pixel values above a predetermined amount.

The ambient light compensated image may include the fluorescence image modified based on the ambient light image.

The ambient light compensated image may include a combination of a reflected light image and the fluorescence image modified to compensate for the contributions of the ambient light to the fluorescence image. Generating the ambient light compensated image may include generating a compensated fluorescence image by subtracting at least a portion of the ambient light image from the fluorescence image; and combining the compensated fluorescence image with the reflected light image.

Generating the compensated fluorescence image may include scaling pixel values of at least one of the ambient light image and the fluorescence image based on at least one of a difference in exposure period and a difference in gain.

Combining the compensated fluorescence image with the reflected light image may include scaling pixel values of at least one of the compensated fluorescence image and the reflected light image based on at least one of a difference in exposure period and a difference in gain.

The one or more programs may include instructions for, in accordance with determining that the level of ambient light in the ambient light image does not meet the threshold, displaying the fluorescence image or an image generated based on the fluorescence image without compensating for the ambient light.

Generating the ambient light compensated image may include scaling pixel values of the ambient light image by a scaling factor that corresponds to an amount that the level of ambient light in the ambient light image is above the threshold.

The threshold may be a lower threshold, and generating the ambient light compensated image may include: scaling pixel values of the ambient light image by a scaling factor when the level of ambient light in the ambient light image is above the lower threshold and below an upper threshold; and not scaling the pixel values of the ambient light image by the scaling factor when the level of ambient light in the ambient light image is above the upper threshold.

The ambient light compensated image may be generated based on an estimated ambient light image that is an estimate of the ambient light at a capture time of the fluorescence image.

The one or more programs may include instructions for displaying the ambient light compensated image. The one or more programs may include instructions for generating and displaying a visual guidance based on the ambient light compensated image.

According to an aspect, a method for combining images captured at different times includes: receiving a time series of images that comprises multiple series of different light source images, the multiple series of different light source images comprising a first light source series and a second light source series, wherein the first light source series comprises a first light source image and the second light source series comprises a second light source image that was captured at a different time than the first light source image; generating at least one estimate of optical flow based on at least one of the multiple series of different light source images; generating, based on the first light source image and the at least one estimate of optical flow, an estimated first light source image that is an estimate of a first light source image captured at a capture time of the second light source image; and generating a combined image based on at least the estimated first light source image and the second light source image.

The first light source image may be an ambient light image, the second light source image may be a fluorescence image, and the estimated first light source image may be an estimated ambient light image. The combined image may be an ambient light compensated fluorescence image or a combination of a reflected light image with the ambient light compensated fluorescence image. The method of may include, prior to generating the estimated ambient light image, determining a level of ambient light in the ambient light image, comparing the level of ambient light in the ambient light image to a threshold, and generating the estimated ambient light image in accordance with the level of ambient light in the ambient light image meeting the threshold.

The first light source image is a fluorescence image, the second light source image is a reflected light image, and the estimated first light source image is an estimated fluorescence image.

Generating the estimated first light source image may include spatially shifting pixel values of the first light source image based on the at least one estimate of optical flow.

Generating the combined image may include subtracting at least a proportion of the estimated first light source image from the second light source image. The time series of images may include a third light source image, and generating the combined image may include combining the third light source image with a result of the subtraction of the estimated first light source image from the second light source image.

A capture time of the first light source image may be between capture times of images of at least one of the multiple series of different light source images used to generate the at least one estimate of optical flow. The at least one estimate of optical flow may be generated based on the second light source image and another image of the second light source series and a capture time of the first light source image may be closer in time to a capture time of the second light source image than a capture time of the other image of the second light source series. Generating the estimated first light source image may include spatially shifting pixel values of the first light source image based on at least one optical flow vector of the at least one estimate of optical flow that is scaled based on a ratio between: (1) a time between the capture time of the second light source image and the capture time of the first light source image, and (2) a time between the images of at least one of the multiple series of different light source images used to generate the at least one estimate of optical flow. The time series of images may include a third light source series, and the at least one estimate of optical flow may include a first estimate of optical flow generated based on the third light source series and a second estimate of optical flow generated based on the second light source series. Generating the estimated first light source image may include spatially shifting pixel values of the first light source image based on a weighted sum of the first and second estimates of optical flow.

The time series of images may include a third light source series, and the at least one estimate of optical flow is generated based on the third light source series. A capture time of the first light source image may be between capture times of images of the third light source series used to generate the at least one estimate of optical flow.

The at least one estimate of optical flow may include multiple flow vectors corresponding to different regions of the multiple images of the time series of images. Generating the estimated first light source image comprises spatially shifting pixel values of different regions of the first light source image based on corresponding flow vectors of the multiple flow vectors.

The at least one estimate of optical flow may include a single flow vector, and generating the estimated first light source image may include spatially shifting the first light source image based on the single flow vector.

The time series of images may include open-field images and the first light source image may include room light.

The time series of images may include endoscopic images and the first light source image may include light from a light source that is not for capturing the time series of images.

The method may include displaying the combined image.

According to an aspect, a system includes one or more processors, memory, and one or more programs stored in the memory for execution by the one or more programs, the one or more programs including instructions for: receiving a time series of images that comprises multiple series of different light source images, the multiple series of different light source images comprising a first light source series and a second light source series, wherein the first light source series comprises a first light source image and the second light source series comprises a second light source image that was captured at a different time than the first light source image; generating at least one estimate of optical flow based on at least one of the multiple series of different light source images; generating, based on the first light source image and the at least one estimate of optical flow, an estimated first light source image that is an estimate of a first light source image captured at a capture time of the second light source image; and generating a combined image based on at least the estimated first light source image and the second light source image.

The first light source image may be an ambient light image, the second light source image may be a fluorescence image, and the estimated first light source image may be an estimated ambient light image. The combined image may be an ambient light compensated fluorescence image or a combination of a reflected light image with the ambient light compensated fluorescence image. The one or more programs may include instructions for, prior to generating the estimated ambient light image, determining a level of ambient light in the ambient light image, comparing the level of ambient light in the ambient light image to a threshold, and generating the estimated ambient light image in accordance with the level of ambient light in the ambient light image meeting the threshold.

The first light source image may be a fluorescence image, the second light source image may be a reflected light image, and the estimated first light source image may be an estimated fluorescence image.

Generating the estimated first light source image may include spatially shifting pixel values of the first light source image based on the at least one estimate of optical flow.

Generating the combined image may include subtracting at least a proportion of the estimated first light source image from the second light source image. The time series of images may include a third light source image, and generating the combined image may include combining the third light source image with a result of the subtraction of the estimated first light source image from the second light source image.

A capture time of the first light source image may be between capture times of images of at least one of the multiple series of different light source images used to generate the at least one estimate of optical flow. The at least one estimate of optical flow may be generated based on the second light source image and another image of the second light source series and a capture time of the first light source image may be closer in time to a capture time of the second light source image than a capture time of the other image of the second light source series. Generating the estimated first light source image may include spatially shifting pixel values of the first light source image based on at least one optical flow vector of the at least one estimate of optical flow that is scaled based on a ratio between: (1) a time between the capture time of the second light source image and the capture time of the first light source image, and (2) a time between the images of at least one of the multiple series of different light source images used to generate the at least one estimate of optical flow. The time series of images may include a third light source series, and the at least one estimate of optical flow may include a first estimate of optical flow generated based on the third light source series and a second estimate of optical flow generated based on the second light source series. Generating the estimated first light source image may include spatially shifting pixel values of the first light source image based on a weighted sum of the first and second estimates of optical flow.

The time series of images may include a third light source series, and the at least one estimate of optical flow may be generated based on the third light source series. A capture time of the first light source image may be between capture times of images of the third light source series used to generate the at least one estimate of optical flow.

The at least one estimate of optical flow may include multiple flow vectors corresponding to different regions of the multiple images of the time series of images. Generating the estimated first light source image may include spatially shifting pixel values of different regions of the first light source image based on corresponding flow vectors of the multiple flow vectors.

The at least one estimate of optical flow may include a single flow vector, and generating the estimated first light source image may include spatially shifting the first light source image based on the single flow vector.

The time series of images may include open-field images and the first light source image may include room light.

The time series of images may include endoscopic images and the first light source image may include light from a light source that is not for capturing the time series of images.

The one or more programs may include instructions for displaying the combined image.

According to an aspect, a non-transitory computer readable storage medium stores one or more programs for execution by a computing system for causing the computing system to perform any of the methods described above.

According to an aspect, a computer program product comprises software code portions for execution by a computing system for causing the computing system to perform any of the methods described above.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an exemplary imaging system for imaging tissue of a subject;

FIG. 2A and FIG. 2B illustrate exemplary timing schemes for acquiring a time series of images that includes both reflected light images and fluorescence images;

FIG. 3 is a flow diagram of an exemplary method for compensating for ambient light in medical imaging based on the level of ambient light;

FIG. 4 is a flow diagram of an exemplary method for combining images of different modalities captured at different times, such as to compensate for motion in the scene or of the camera relative to the scene that may occur between the different times;

FIG. 5 illustrates an example of generating an ambient light compensated fluorescence image using an estimate of optical flow generated from ambient light images;

FIG. 6 illustrates an example of generating an ambient light compensated fluorescence image using an estimate of optical flow generated from reflected light images and, optionally, an estimate of optical flow generated from ambient light images;

FIG. 7 illustrates an example of combining fluorescence light and reflected light images; and

FIG. 8 is a functional block diagram of an exemplary computing system.

### DETAILED DESCRIPTION

In the following description of the various examples, reference is made to the accompanying drawings, in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art and the generic principles herein may be applied to other examples. Thus, the present invention is not intended to be limited to the examples shown but is to be accorded the widest scope consistent with the principles and features described herein.

According to an aspect, systems and methods described herein include selectively performing ambient light compensation for fluorescence images in order to allow an imaging sensor to image fluorescence light having a waveband that overlaps with ambient light while avoiding the introduction of noise when ambient light compensation is not needed. When the waveband of fluorescence light captured by one or more imaging sensors overlaps that of ambient light, sufficiently high ambient light levels can lead to lack of contrast between regions of high and low fluorescence light emission in fluorescence images, which reduces the usefulness of the fluorescence images as a clinical tool. Ambient light compensation can reduce at least some of the effects of ambient light on the fluorescence light images, improving the contrast of the fluorescence images. However, ambient light compensation can introduce noise to the fluorescence light images. At times when there is little or no ambient light (for example, the room lights are turned off or substantially dimmed for open-field imaging or there is no source of light pollution in a surgical cavity during endoscopic imaging), not performing ambient light compensation can provide the highest quality fluorescence images, with the lowest noise. The systems and methods described herein include determining the level of ambient light and performing ambient light compensation only when the level is sufficiently high, thus improving contrast at times of relatively high ambient light levels and avoiding the introduction of noise at times of relatively low ambient light levels.

An imaging system is configured to capture ambient light images in alternating fashion with fluorescence light images (and, optionally, other types of images) so that the ambient light images can be used to compensate for contributions of ambient light to the fluorescence images. For a given fluorescence image, a corresponding ambient light image is analyzed to determine a level of ambient light intensity. The level of ambient light intensity is compared to a threshold and, if it meets the threshold, the ambient light image is used to compensate for the contribution of ambient light to the fluorescence image. If the level of ambient light does not meet the threshold, which can be set to correspond to a level of ambient light that likely has an acceptably small effect, if any, on the fluorescence image, then the fluorescence image may be used without performing ambient light compensation. This can avoid unnecessarily introducing noise into a fluorescence image arising from the ambient light compensation process.

The amount of ambient light compensation can be scaled based on the level of ambient light intensity. For example, a second threshold for ambient light intensity, which is higher than the threshold used for determining whether or not to perform ambient light compensation, can be defined and ambient light compensation can be scaled according to where the level of ambient light intensity falls between the two thresholds. This can avoid abrupt changes in the fluorescence images when the level of ambient light intensity is near the threshold that determines when ambient light compensation is performed. Ambient light compensation can include other scaling, including scaling based on differences in exposure time and levels of gain between the ambient light and fluorescence light images

In addition to introducing noise, ambient light compensation can create artifacts arising from motion of the camera or in the imaged scene. This is due to the difference in capture time between the fluorescence light image and the ambient light image used for ambient light compensation for the fluorescence light image. For example, in ambient light compensation, since the brightness at a given pixel location during the fluorescence frame may no longer correspond to the same location in space during the ambient light frame because of motion occurring between their respective capture times, artifacts may be produced, such as edges of objects in the scene appearing as phantom fluorescence. This effect can arise in other contexts as well, such as when combining reflected light images and fluorescence images. According to another aspect, systems and methods described herein can use motion compensation techniques to reduce the effects of motion when combining images corresponding to different light sources, such as when using ambient light images to compensate for the effects of ambient light in fluorescence images.

An imaging system may capture a time series of images that includes series of different light source images, including a series of first light source images and a series of second light source images, with the images of the first and second light source series being captured in alternating fashion with one another (in addition to one or more other light source images). At least one estimate of optical flow can be determined based on one or more of the series of different light sources images. The estimate of optical flow is used to generate an estimate of an image of the first light source series captured at a capture time of an image of the second light source series. In other words, the estimate of the image of the first light source series estimates what a first light source series image would have been had it been captured simultaneously with the image of the second light source series. The estimate of the image of the first light source series can then be combined with the image of the second light source image.

Common techniques for image stabilization include identification of optical flow and then application of a transform to the image in order to reduce shakiness or other motion effects. Such techniques or aspects of such techniques can be employed by the systems and methods described here. For example, an estimate of optical flow may be calculated using the Lukas-Kanade method or by any other suitable method. The estimate of optical flow can include an optical flow vector for a selected number of points within an image. The points at which this is calculated may be determined by a feature detection algorithm or the points may be randomly or evenly spaced across the image. Optical flow vectors from the selected points may be combined by a voting method (e.g., reject outliers, then average the most common values) to create a single motion vector for the image, which may be a simple translation, or may also include rotation and zoom. Pixel data of an image, such as an ambient light image, may be shifted according to the motion vector in order to generate an estimate of what an ambient light frame would have been at a different point in time, such as at the time that a fluorescence frame was acquired. The motion vector used for each point in the image may either be a single vector for the whole image, such as based on the most common motion detected, or may be a more localized vector based on the motion vectors calculated near the point being processed.

The estimate of optical flow can be generated from the same series of images from which the estimated image is generated, from the same series of images that the estimated image is combined with, or from some other series of images. For example, for ambient light compensation, an estimated ambient light image for combining with a fluorescence light image can be generated based on an estimate of optical flow generated from ambient light images, from fluorescence images, and/or from reflected white light images.

In the above example, the ambient light frames and/or the fluorescence frames may be fairly dim, making the calculation of optical flow difficult and inaccurate. Accordingly, the estimate of optical flow may be generated from surrounding white light frames generated when performing combined reflected light and fluorescence imaging. Optionally, multiple estimates of optical flow may be used, each being generated from a different series of images.

As noted above, these techniques are not limited to ambient light compensation. Fluorescence is often displayed as an overlay on a reflected white light or other reflected light image. Motion can result in the overlay being displayed in a slightly different position than the underlying tissue. Accordingly, systems and methods may be configured to perform the motion compensation techniques described above when combining fluorescence and reflected light images. For example, an estimate of optical flow can be generated from the reflected light images and used to generate an estimate of a florescence image at a capture time of a reflected light image. The estimated fluorescence light image and the reflected light image can then be combined while reducing the introduction of motion artifacts.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

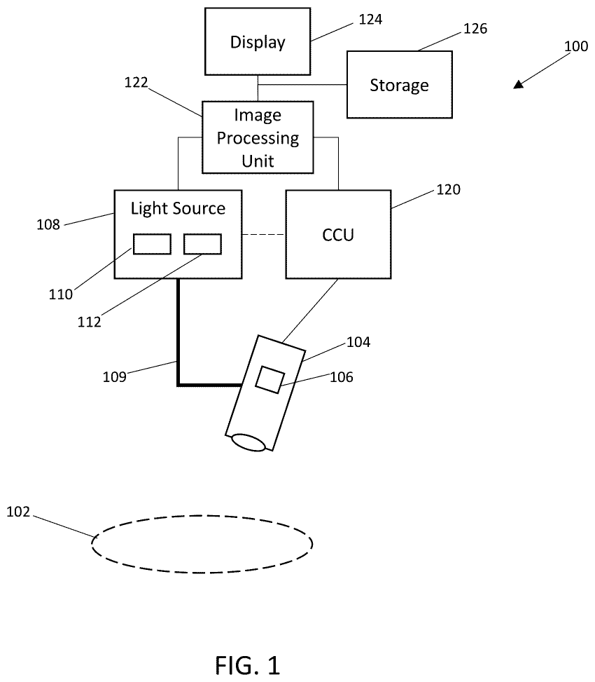

FIG. 1 illustrates an exemplary imaging system 100 for imaging tissue 102 of a subject during an imaging session, a surgical procedure, or a non-surgical medical procedure. System 100 include an image acquisition assembly 104 (also referred to herein as an imager) that has at least one image sensor 106 configured to capture an image or a sequence of video frames depicting the tissue and/or one or more features of the tissue. The image acquisition assembly 104 can be a hand-held device, such as an open-field camera or an endoscopic camera, or can be mounted or attached to a mechanical support arm.

The image acquisition assembly 104 may be connected to a camera control unit (CCU) 120, which may generate one or more single snapshot images and/or video frames (referred to herein collectively as images) from imaging data generated by the image acquisition assembly 104. The images generated by the camera control unit 120 may be transmitted to an image processing unit 122 that may apply one or more image processing techniques described further below to the images generated by the camera control unit 120. Optionally, the camera control unit 120 and the image processing unit 122 are integrated into a single device. The image processing unit 122 (and/or the camera control unit 120) may be connected to one or more displays 124 for displaying the one or more images generated by the camera control unit 120 or one or more images or other visualizations generated based on the images generated by the camera control unit 120. The image processing unit 122 (and/or the camera control unit 120) may store the one or more images generated by the camera control unit 120 or one or more images or other visualizations generated based on the images generated by the camera control unit 120 in one or more storage devices 126. The one or more storage devices can include one or more local memories, one or more remote memories, a recorder or other data storage device, a printer, and/or a picture archiving and communication system (PACS). The system 100 may additionally or alternatively include any suitable systems for communicating and/or storing images and image-related data.

The imaging system 100 may include a light source 108 configured to generate light that is directed to the field of view to illuminate the tissue 102. Light generated by the light source 108 can be provided to the image acquisition assembly 104 by a light cable 109. The image acquisition assembly 104 may include one or more optical components, such as one or more lenses, fiber optics, light pipes, etc., for directing the light received from the light source 108 to the tissue. The image acquisition assembly 104 may be an endoscopic camera that includes an endoscope that includes one or more optical components for conveying the light to a scene within a surgical cavity into which the endoscope is inserted. The image acquisition assembly 104 may be an open-field imager and may include one or more lenses that direct the light toward the field of view of the open-field imager.

The light source 108 includes one or more visible light emitters 110 that emit visible light in one or more visible wavebands (e.g., full spectrum visible light, narrow band visible light, or other portions of the visible light spectrum). The visible light emitters 110 may include one or more solid state emitters, such as LEDs and/or laser diodes. The visible light emitters 110 may include blue, green, and red (or other color components) LEDs or laser diodes that in combination generate white light or other illumination for reflected light imaging. These color component light emitters may be centered around the same wavelengths around which the image acquisition assembly 104 is centered. For example, in variations in which the image acquisition assembly 104 includes a single chip, single color image sensor having an RGB color filter array deposited on its pixels, the red, green, and blue light sources may be centered around the same wavelengths around which the RGB color filter array is centered. As another example, in variations in which the image acquisition assembly 104 includes a three-chip, three-sensor (RGB) color camera system, the red, green, and blue light sources may be centered around the same wavelengths around which the red, green, and blue image sensors are centered.

The light source 108 can include one or more excitation light emitters 112 configured to emit excitation light suitable for exciting intrinsic fluorophores and/or extrinsic fluorophores (e.g., a fluorescence imaging agent that has been introduced into the subject) located in the tissue being imaged. The excitation light emitters 112 may include, for example, one or more LEDs, laser diodes, arc lamps, and/or illuminating technologies of sufficient intensity and appropriate wavelength to excite the fluorophores located in the object being imaged. For example, the excitation light emitter(s) may be configured to emit light in the near-infrared (NIR) waveband (such as, for example, approximately 805 nm light), though other excitation light wavelengths may be appropriate depending on the application.

The light source 108 may further include one or more optical elements that shape and/or guide the light output from the visible light emitters 110 and/or excitation light emitters 112. The optical components may include one or more lenses, mirrors (e.g., dichroic mirrors), light guides and/or diffractive elements, e.g., so as to help ensure a flat field over substantially the entire field of view of the image acquisition assembly 104.

The image acquisition assembly 104 may acquire reflected light images based on visible light that has reflected from the tissue, and/or fluorescence images based on fluorescence emitted by fluorophores in the tissue that are excited by the fluorescence excitation light. The at least one image sensor 106 may include at least one solid state image sensor. The at least one image sensor 106 may include, for example, a charge coupled device (CCD), a CMOS sensor, a CID, or other suitable sensor technology. The at least one image sensor 106 may include a single image sensor (e.g., a grayscale image sensor or a color image sensor having an RGB color filter array deposited on its pixels). The at least one image sensor 106 may include three sensors, such as one sensor for detecting red light, one for detecting green light, and one for detecting blue light.

The camera control unit 120 can control timing of image acquisition by the image acquisition assembly 104. The image acquisition assembly 104 may be used to acquire both reflected light images and fluorescence images and the camera control unit 120 may control a timing scheme for the image acquisition assembly 104. The camera control unit 120 may be connected to the light source 108 for providing timing commands to the light source 108. Alternatively, the image processing unit 122 may control a timing scheme of the image acquisition assembly 104, the light source 108, or both.

The timing scheme of the image acquisition assembly 104 and the light source 108 may enable separation of the image signal associated with the reflected light signal and the image signal associated with the fluorescence signal. In particular, the timing scheme may involve illuminating the tissue with illumination light and/or excitation light according to a pulsing scheme, and processing the reflected light image signal and fluorescence image signal with a processing scheme, wherein the processing scheme is synchronized and matched to the pulsing scheme to enable separation of the two image signals in a time-division multiplexed manner. Examples of such pulsing and image processing schemes have been described in U.S. Pat. No. 9,173,554, filed on Mar. 18, 2009 and titled “IMAGING SYSTEM FOR COMBINED FULL-COLOR REFLECTANCE AND NEAR-INFRARED IMAGING,” the contents of which are incorporated in their entirety by this reference. However, other suitable pulsing and image processing schemes may be used to acquire reference video frames and low light video frames simultaneously, for example to acquire reflected light video frames and fluorescence video frames simultaneously.

In some variations, the system 100 may include image stabilizing technology that helps compensate for some ranges of motion (e.g., caused by unsteady hands holding the image acquisition assembly) in the acquired images. The image stabilizing technology may be implemented in hardware, such as with optical image stabilization technology that counteracts some relative movement between the image acquisition assembly and the object by varying the optical path to the image sensor (e.g., lens-based adjustments and/or sensor-based adjustments). Additionally, or alternatively, the image stabilization technology may be implemented in software, such as with digital image stabilization that counteracts some relative movement between the image acquisition assembly and the object (e.g., by shifting the electronic image between video frames, utilizing stabilization filters with pixel tracking, etc.). Such image stabilizing technology may, for example, help correct for motion blur in the characteristic low light video output (or in the acquired low light video frames) resulting from relative motion during long exposure periods.

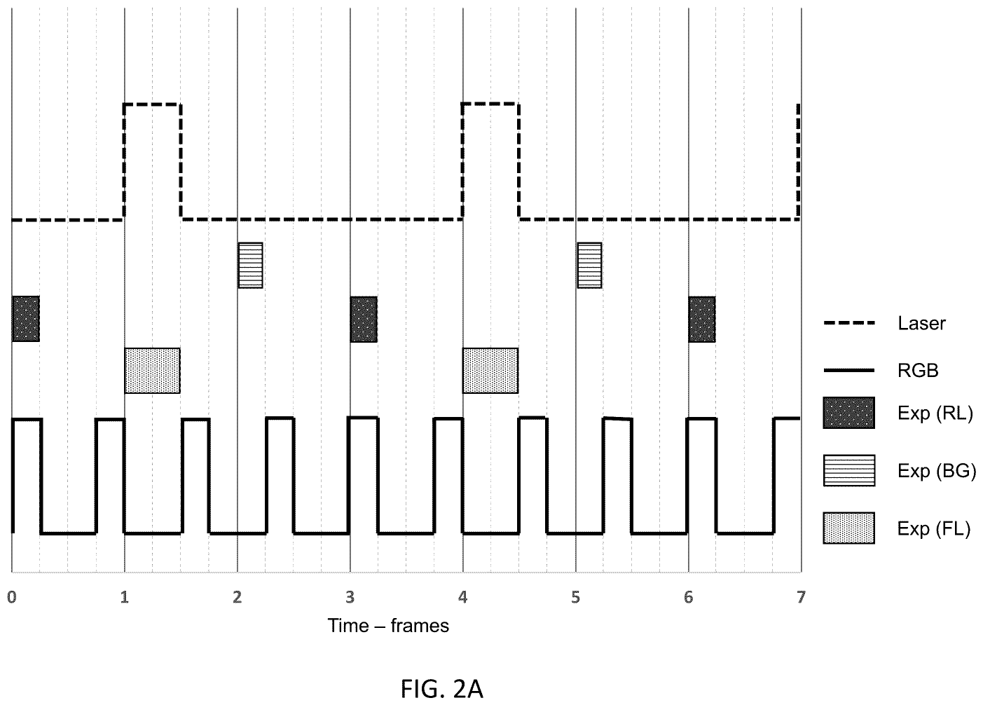

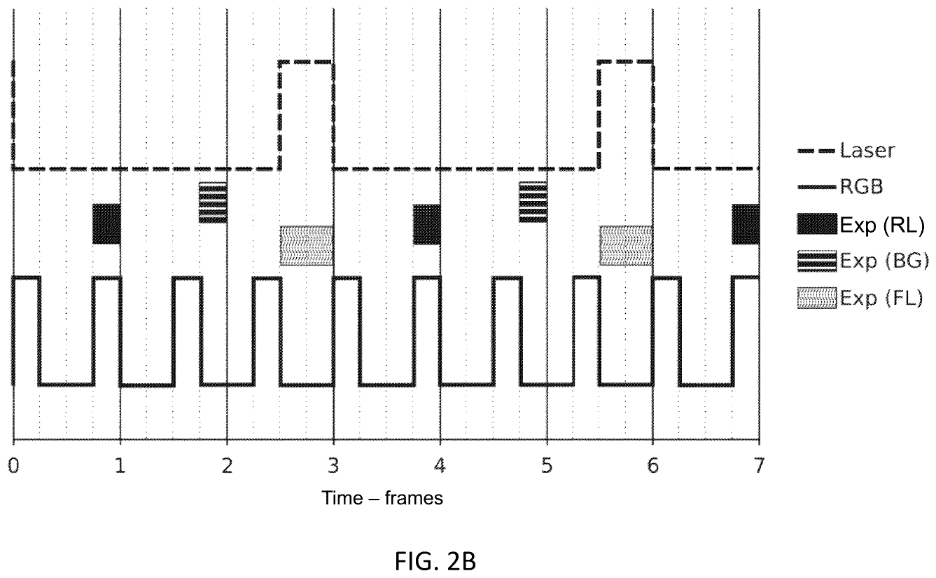

FIG. 2A illustrates an exemplary timing scheme that can be implemented by system 100 for acquiring a time series of images that includes both reflected light images and fluorescence images. The exemplary timing scheme includes visible light (e.g., RGB) illumination and fluorescence excitation (e.g., Laser) illumination of a scene, and imaging sensor exposures for reflected (RL) and fluorescence (FL) that are configured to allow removal of contributions of ambient light from the fluorescence image signal. Exposures for reflected light and fluorescence light are shown in sequence along with an exposure to capture the background (BG) image signal due to ambient light. The numbers along the axis represent frames acquired by the image acquisition assembly 104. Pulsing of visible illumination light is shown by the solid line, and pulsing of fluorescence excitation light is shown by the dashed line.

During frame 1, the one or more image sensors 106 are exposed during the period of the first visible light pulse such that the one or more image sensors 106 acquire an image from visible light reflected from the field of view of the image acquisition assembly 104. Thus, frame 1 is a reflected light frame. During frame 2, the one or more image sensors 106 are exposed during the period of the fluorescence excitation light pulse. During this period, the visible light is not provided such that the one or more image sensors 106 acquire an image of fluorescence light emitted by tissue within the field of view of the image acquisition assembly 104.

During frame 3, the one or more image sensors 106 are exposed during a period in which neither the visible light nor the fluorescence excitation light is provided to the scene. Thus, any light captured by the one or more image sensors 106 during frame 3 is light from the scene that is not generated by the light source 108. This light is referred to herein as ambient light or background light and represents any light from the scene that is not provided by the light source 108. The image acquisition assembly 104 can be an open-field imager imaging an open surgical field and the light captured by the image acquisition assembly 104 can be light coming from one or more room lights in the room where the imaging is being conducted that reflects off of the field. The image acquisition assembly 104 can be an endoscopic imager, and the ambient/background light can be light coming from a lighted stent or other light delivery device present in the surgical cavity, or the ambient/background light can be light generated by a cauterizing tool or other tool that generates light during its use. Light generated by such external sources (external to the imaging system 100) that reflects off of the scene can be captured during frame 3. Thus, frame 3 is an ambient light (background light) frame.

An exemplary timing scheme for the frames of FIG. 2A captured at 60 Hz may include pulsing the visible light illumination at 80 Hz. The fluorescence excitation illumination may be pulsed at 20 Hz and the pulse duration or width may be increased, e.g., up to double the white light pulse duration, to enable a longer corresponding fluorescence exposure. The longer duration can increase the signal strength of the fluorescence exposure to help compensate for the relatively low intensity of fluorescence light emitted from the scene.

The timing scheme of FIG. 2A is merely exemplary and it will be understood by a person having ordinary skill in the art that the frequency of the visible light illumination and the Laser light illumination pulses can be suitably adjusted based on a desired frame rate and/or a frame rate capability of the image acquisition assembly 104. Further, the durations of the visible light illumination and the Laser light illumination pulses can be selected as desired and/or the durations of the reflected light, fluorescence light, and ambient light exposures can be selected as desired. Optionally, multiple types of fluorescence light images can be captured, including fluorescence images for different wavebands of fluorescence light generated by different types of fluorophores excited by different fluorescence excitation lights. The particular order in which the reflected light, fluorescence light, and ambient light frames (and any other types of frames) are captured can be selected as desired. An example of another timing scheme that may be used according to the principles described herein is illustrated in FIG. 2B. Other suitable timing schemes are described in U.S. Pat. No. 11,140,305 (“Open-Field Handheld Fluorescence Imaging Systems and Methods”), issued Oct. 5, 2021, the entire contents of which are hereby incorporated by reference.

The pattern of illumination pulses and exposures can repeat continuously for at least a portion of an imaging session, resulting in a time series of images (e.g., frames 1-7 of FIG. 2A) that include multiple series of different light source images. A first series of the multiple series of different light source images is reflected light images (e.g., frames 1, 4, and 7 of FIG. 2A) resulting from a visible light source illuminating a scene and visible light reflecting from the scene. A second series of the multiple series of different light source images is fluorescence images (e.g., frames 2 and 5 of FIG. 2A) resulting from fluorescence excitation light illuminating the scene and fluorescence light being emitted by fluorophores in the scene that are excited by the fluorescence excitation light. A third series of the multiple series of different light source images is ambient light images (e.g., frames 3 and 6 of FIG. 2A) resulting from any light illuminating the scene that is not purposefully illuminating the scene for generating the images, such as room light or light pollution from light sources that are not intended for illuminating the scene for imaging.

Ambient light that illuminates a scene may include wavelengths that are detectable by one or more of the at least one image sensor 106 of the image acquisition assembly 104 that are used to capture the fluorescence light images such that ambient light contributes to the image signal of the fluorescence light images. The ambient light images can be used to remove contributions of ambient light to the fluorescence images. However, in instances in which relatively little or no ambient light is contributing to the fluorescence images (such as when the room lights are sufficiently dimmed or turned off during open-field imaging or during endoscopic imaging when there is no other light source present in the surgical cavity), the process for removing ambient light from the fluorescence images may result in increased noise without noticeably improving contrast. In such instances, it may be preferable not to compensate for ambient light in the fluorescence images.

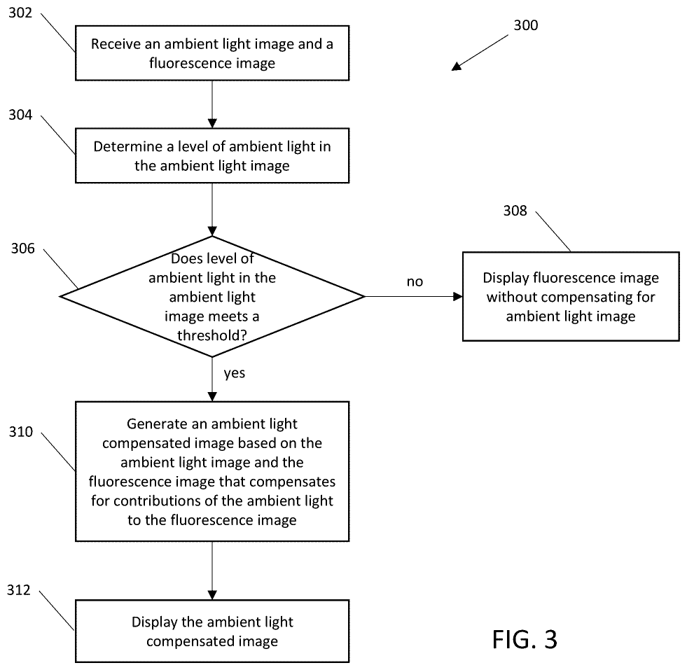

FIG. 3 is a flow diagram of a method 300 for compensating for ambient light in medical imaging based on the level of ambient light. Method 300 can be used to compensate for the contribution of ambient light in fluorescence light images when the level of ambient light is sufficiently high but not compensate for ambient light when the level of ambient light is sufficiently low. Method 300 may be performed by system 100 of FIG. 1.

At step 302, an ambient light image and a fluorescence image are received at a computing system. For example, with reference to FIG. 1, an ambient light image and a fluorescence light image acquired by image acquisition assembly 104 may be received at image processing unit 122 from CCU 120, either directly or from a memory to which the images have been saved. The ambient light image and fluorescence image may be part of a time series of images received at the computing system. For example, the ambient light image and fluorescence image may be frames of video that are captured closed in time to one another. Method 300 may be performed on multiple sets of ambient light and fluorescence light images in the series of images, such as on each set of ambient light and fluorescence light images in the series of images or on sets of ambient light and fluorescence light images at regular intervals (e.g., every other set, every third set, every fourth set, etc.).

At step 304, a level of ambient light is determined from the ambient light image. The level of ambient light can be determined in any suitable fashion. For example, the level of ambient light can be determined by generating a histogram of the ambient light image and determining the number or proportion of pixels having an intensity value that is above a predetermined intensity. Alternatively, the level of ambient light can be the average pixel intensity in at least a portion of the ambient light image or the maximum pixel intensity in at least a portion of the ambient light image. The level of ambient light can be a quantitative value, such as the percentage of pixels that have an intensity value that is above a predetermined intensity.

At step 306, a determination is made whether the level of ambient light from the ambient light image meets a threshold. For example, the level of ambient light can be compared to the threshold to determine whether the level of ambient light is at or above below the threshold. The threshold can be selected such that, when the ambient light level meets the threshold, it is expected that ambient light has some noticeable effect on the fluorescence image and, when the ambient light level does not meet the threshold, it is expected that ambient light has either had no noticeable effect on the fluorescence image or has had an acceptable effect on the fluorescence image. The threshold may be, for example, a predetermined proportion of the ambient light image that has pixel intensity values that are about a predetermined amount. For example, the threshold may be 20 percent of the pixels of the ambient light image that have intensity values that are above 30 (e.g., for a 256 intensity value scale), such that an ambient light image having a level of ambient light of 21 percent of its pixels that have an intensity above 30 meets the threshold, whereas an ambient light image having a level of ambient light of 19 percent of its pixels that have an intensity above 30 does not meet the threshold.

If the level of ambient light from the ambient light image does not meet the threshold as determined in step 306, then at step 308, the fluorescence image may be used without correcting for ambient light. For example, the fluorescence image may be displayed on one or more displays, such as display 124 of FIG. 1. Additionally or alternatively, the fluorescence image may be combined with another image, such as with a reflected light image (e.g., an image generated when a scene is illuminated with reflected light such as white light) to produce a combination reflected light and fluorescence light image, which may be displayed on one or more displays.

If the level of ambient light from the ambient light image does meet the threshold as determined in step 306, then at step 310, an ambient light compensated image is generated based on the fluorescence image and the ambient light image such that the contribution of ambient light to the fluorescence image is compensated for. For example, an ambient light compensated image may be generated in which pixel values from the ambient light image are subtracted (e.g., pixel-by-pixel) from the fluorescence image pixel values. The pixel values of the ambient light image may be scaled by a scaling factor prior to subtraction to account for differences in exposure time and/or gain between the ambient light image and the fluorescence image. The scaling factor can be or include, for example, a ratio of exposure times and/or a ratio of gains associated with the ambient light image and the fluorescence image. For example, with reference to the timing scheme of FIG. 2A where the fluorescence exposure time is about twice that of the ambient light exposure time (one-half of a frame versus one-quarter of a frame), the pixel values of the ambient light image may be multiplied by a scaling factor of two before being subtracted from the ambient light frame. A scaling factor associated with differences in exposure time and/or gain for an ambient light image BG and a fluorescence image FL can be defined as:

$$G = \frac{{Exp}_{F L}}{{Exp}_{B G}} \times \frac{{Gain}_{F L}}{{Gain}_{B G}}$$

where G is an “effective gain” scaling factor, Exp is the exposure time for the respective image, and Gain is the gain for the respective image.

The ambient light compensated image may be an ambient light compensated fluorescence image generated by subtracting the ambient light image (scaled or unscaled) from the fluorescence image. The ambient light compensated image may be a combination of a reflected visible light image (e.g., a reflected light image) and the fluorescence image compensated based on the ambient light image. For example, a combination reflected light and fluorescence image may be generated by subtracting scaled or unscaled pixel values of the ambient light image from pixel values of the fluorescence image and adding those values (also scaled or unscaled depending on difference in gain and/or exposure time relative to the reflected light image, such as using the effective gain scaling factor described above) to pixel values from the reflected light image. Ambient light compensation may also be performed for the reflected light image.

Method 300 may include an optional step 312 of displaying the ambient light compensated image. For example, an ambient light compensated fluorescence image or an ambient light compensated combined reflected light and fluorescence image may be displayed on one or more displays (e.g., display 124 of FIG. 1) during a medical procedure. Additionally or alternatively, the ambient light compensated image can be further processed, such as to assess one or more characteristics of anatomy of a patient captured in the ambient light compensated image. For example, an ambient light compensated fluorescence image may be analyzed to quantify an amount of perfusion of tissue based on an intensity of the fluorescence in the ambient light compensated fluorescence image. The results of any assessment may be provided to a user in a visual guidance with or without displaying the ambient light compensated image. For example, one or more numerical values associated with the assessment (e.g., a quantification of degree of perfusion) may be displayed to the user as text overlaid on a reflected light image or on a combined reflected light and fluorescence image generated from the ambient light compensated fluorescence image, or the results of the assessment may be used to generate a false color overlay on a reflected light image that indicates a degree of perfusion of the tissue.

According to method 300, ambient light compensation for fluorescence light images is performed when the level of ambient light in an ambient light image is sufficiently high and is not performed when the level of ambient light is sufficiently low. Not performing compensation when the ambient light is sufficiently low can be advantageous because the compensation process performed when the ambient light is low may increase the noise in the resulting image and/or may create artifacts if there is motion in the time between when the fluorescence image is captured and when the ambient light image is captured. An imaging system performing method 300 automatically performs ambient light compensation when needed without requiring a user to select whether or not to perform ambient light compensation, which may be difficult for a user to know when to do. Method 300 can be performed on images of a video stream (e.g., on each set of ambient light and fluorescence light frames) such that ambient light compensation is performed for the video stream. It may be desirable for the user to be informed of when the ambient light compensation is being performed, and therefore, method 300 may include providing a notification that ambient light compensation has been performed for a given image. Any suitable notification could be used, including, for example, a graphical indication (e.g., an icon or text) overlayed on a display generated based on the ambient light compensated image.

When the level of ambient light associated with a series of fluorescence images (e.g., a video stream) is near the threshold described above with respect to step 306, ambient light compensation may be performed for some of the fluorescence images but not others. To avoid abrupt changes between an ambient light compensated image and a non-ambient light compensated image that could occur when they are generated close in time, method 300 can include scaling the amount of ambient light compensation based on the level of ambient light determined at step 304. So, for example, a level of ambient light that is nearer the threshold of step 306 may result in less compensation than a level of ambient light that is further from the threshold of step 306.

The scaling of the ambient light image based on the ambient light level can include multiplying intensity values of the ambient light image by a scaling factor that is based on the ambient light level determined in step 304. This scaling factor can be in addition to any scaling factor associated with exposure time and/or gain. The scaling factor may be applied over a range of ambient light levels that includes the threshold of step 306 at the low end and a second threshold at a high end. Ambient light images associated with ambient light levels that are above the second threshold may not be scaled by the scaling factor (or the scaling factor may be set to 1).

Between the lower and upper thresholds, the scaling factor may be any suitable function of the ambient light level. For example, the scaling factor can be a linear function that is a ratio between the difference between: (a) the ambient light level determined at step 304 and the lower threshold, and (b) the difference between the upper threshold and the lower threshold, such as:

$$S = \frac{L - X}{Y - X}$$

where, L is the level of ambient light determined at step 304, X is the lower threshold used in step 306 and Y is the upper threshold. This scaling factor will range from 0 (when the ambient light level is equal to the lower threshold) to 1 (when the ambient light level is equal to the higher threshold).

As noted above, the scaling factor associated with the level of ambient light (e.g., S above) can be used in combination with a scaling factor associated with differences in exposure time and/or gain (the effective gain G above). With these two factors, an ambient light compensated fluorescence image for ambient light levels that are in the range of the lower threshold X and the upper threshold Y can be generated based on ambient light image BG and a fluorescence image FL as follows:

$$F {L}_{c} = F L - ( S \cdot G \cdot BG )$$

where, FL, is the ambient light compensated fluorescence image. As noted above, such an ambient light compensated fluorescence image can be displayed or further processed or can be combined with another image, such as a reflected light image, for display.

As explained above with respect to the timing sequence of FIG. 2A, an ambient light image may be captured at a different time than a fluorescence image. For example, if the ambient light image and the fluorescence image are captured back-to-back, the difference between the times of capture can be the reciprocal of the frame rate. There may be motion that occurs during this time such that a given location of the scene that is captured by a particular region of pixels in, say, the ambient light image, may be captured by a different region of pixels in the fluorescence image. A straight subtraction of these two images may produce motion artifacts associated with the motion that occurred between when the ambient light image and the fluorescence image were captured. Described in detail below is a motion compensation process that can be used to compensate for motion when generating an ambient light compensated image.

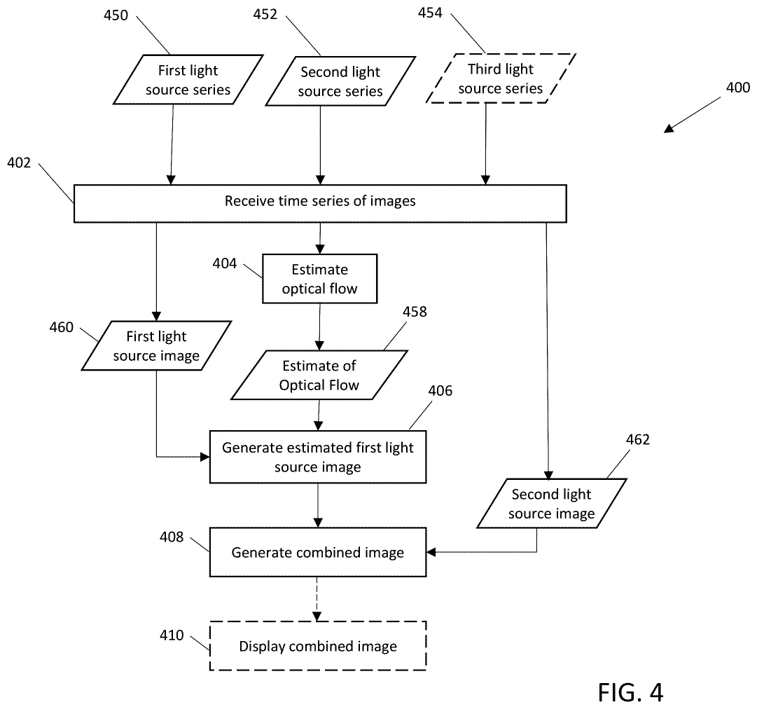

FIG. 4 is a flow diagram of a method 400 for combining images of different modalities captured at different times, such as to compensate for motion in the scene that may occur between the different times. Method 400 can be used, for example, in conjunction with method 300 to combine an ambient light image with a fluorescence image to generate an ambient light compensated fluorescence image while minimizing or eliminating motion-based artifacts. However, method 400 is not limited to use in conjunction with method 300. For example, method 400 can be used to combine a fluorescence image (ambient light compensated or not) with a reflected light image. Method 400 may be performed by any suitable computing system, such as image processing unit 122 of system 100 of FIG. 1.

At step 402, a time series of images is received at the computing system. The time series of images can include series of different light source images captured by an imaging system, such as using image acquisition assembly 104 of imaging system 100. For example, the time series of images can include the sequence of frames of FIG. 2A. The steps below are described with respect to a set of images of the time series of images but it should be understood that the steps can be performed repeatedly for each set of images, such as on sequential sets of frames of a video as the video is generated during an imaging session.

The time series of images includes a first light source series 450 that includes images captured that are associated with a first light source and a second light source series 452 that includes images captured that are associated with a second light source. The first light source could be, for example, a fluorescence excitation light source and the images of the first light source series can be images captured from the fluorescence light emitted from the scene. The second light source could be, for example, ambient light and the images of the second light source series may be ambient light images captured when the scene is not illuminated by the imaging system. The images of the first light source series are captured sequentially in time with respect to each other and with respect to corresponding images of the second light source series. Thus, an image of the first light source series was captured before an image of the second light source series, which was captured before the next image of the first light source series, which was captured before the next image of the second light source series, and so on. The first light source series can be a first set of frames of a video and the second light source series can be a second set of frames of the video. For example, with reference to FIG. 2A, the first light source series can include the fluorescence exposure frames 2 and 5 and the second light source series can include the ambient light exposure frames 3 and 6. The times series of images can include other light source series, such as a third light source series 454, which could include images captured from reflected visible light when the scene is illuminated by a visible light source of the imaging system.

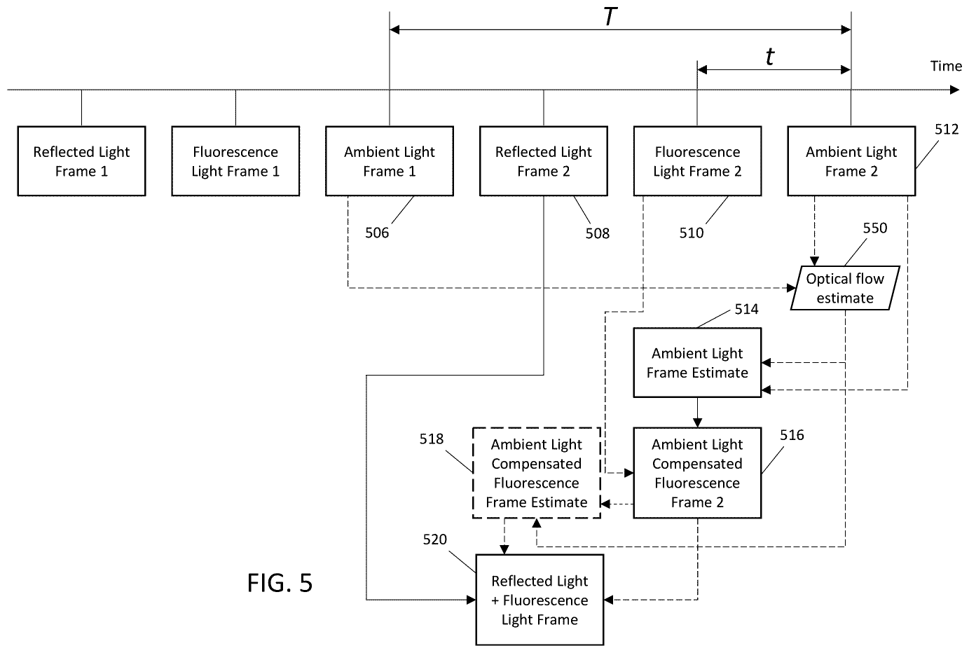

An example of a time series of images that may be received at step 402 is illustrated in FIG. 5, which illustrates the generation of an ambient light compensated fluorescence image. In the example of FIG. 5, the time series of images includes three different light source series-a reflected light series that includes reflected light frames captured when white light is illuminating a scene, a fluorescence light series that includes fluorescence light frames captured when fluorescence excitation light is illuminating a scene and the white light is not illuminating the scene, and an ambient light series that includes images captured when the scene is not being illuminated and any light captured is associated with ambient light. One frame of each series is shown in the example of FIG. 5 for simplicity.

At step 404, an estimate of optical flow 458 is generated based on the time series of fluorescence images. The estimate of optical flow 458 can be generated from at least one of the different light source series. For example, the estimate of optical flow 458 can be generated from the first light source series 450, the second light source series 452, or the third light source series 454. Step 404 can include generating multiple estimates of optical flow, each generated from a different light source series.

Generally speaking, the estimate of optical flow 458 is an estimate of how the scene has moved relative to the imager between two images and is commonly used in image stabilization techniques. An estimate of optical flow may indicate how far and in what direction to shift pixels of one image of a scene to align with pixels of another image of the scene. The motion between images that may be estimated by the estimate of optical flow may be due to movement in the scene and/or due to movement of the imager.

The estimate of optical flow 458 can be generated based on two sequential images in the given light source series from which the estimate is generated. In the example of FIG. 5, the estimate of optical flow (optical flow estimate 550) is generated from sequential ambient light images-ambient light frame 1 506 and ambient light frame 2 512. The estimate of optical flow 458 may be calculated using, for example, the Lukas-Kanade method, or by any other method, such as methods commonly used in image stabilization algorithms. Generating the estimate of optical flow 458 may include generating optical flow vectors for a selected number of points within an image. The points at which the optical flow vectors are calculated may be determined by a feature detection algorithm, or the points may be randomly or evenly spaced across the image. The estimate of optical flow can be the set of optical flow vectors. Generating the estimate of optical flow 458 may include combining multiple optical flow vectors from selected points, such as by a voting method (for example, rejecting outliers and then averaging the most common values) to create a single vector for the image or for a portion of the image. The single vector may correspond to just a translation or may also include rotation and/or zoom.

At step 406, a first light source image 460 from the first light source series 450 is used along with the estimate of optical flow 458 to generate an estimated first light source image. The estimated first light source image is an estimate of a first light source image captured at a capture time of a second light source image 462 of the second light source series 452. In other words, pixel values of the first light source image 460 are spatially shifted based on the estimate of optical flow to generate an estimate of what the first light source image would have been had it been captured at the same time as the second light source image 462. As explained further below, this enables the second light source image to be combined with the estimate of the first light source image without introducing (or with minimal introduction of) motion artifacts resulting from the fact that the first and second light sources images were captured at different times.

In the example of FIG. 5, which illustrates aspects of generating an ambient light compensated fluorescence image (such as at step 310 of method 300), the estimate of optical flow 550 is used in combination with ambient light frame 2 512 (the first light source image in the example of FIG. 5) to generate an ambient light frame estimate 514 (the estimated first light source image) that aligns in time with fluorescence light frame 2 510 (the second light source image in the example of FIG. 5). Since the estimate of optical flow 550 in FIG. 5 estimates the shift in the scene from the time that ambient light frame 1 506 was captured to the time that ambient light frame 2 512 was captured (duration T), but ambient light frame 2 512 need only be shifted for the time difference between when ambient light frame 2 512 was captured and when fluorescence light frame 2 510 was captured (a shorter duration 1), the estimate of optical flow 550 may be scaled according to the differences in duration T and duration t for generating the ambient light frame estimate 514. The estimate of optical flow 550 may be scaled, for example, by the ratio of the duration/between when the fluorescence light frame 2 510 is captured and when the ambient light frame 2 512 and the duration T between when the ambient light frame 1 506 was captured and when the ambient light frame 2 512 is captured. For example, if the estimate of optical flow 550 (from ambient light frame 1 506 to ambient light frame 2 512) is a shift to the right of 10 pixels, duration Tis 10 ms and duration t is 2 ms, then scaling the estimate of optical flow 550 can include multiplying the 10 pixels by 2 divided by 10, resulting in 2 pixels of shift, which would be applied to shift the pixels of ambient light frame 2 to the left.

In the example of FIG. 5, ambient light frame 2 512 was used to generate the ambient light frame estimate 514 because ambient light frame 2 512 was captured closer in time to fluorescence light frame 2 510 and, therefore, may be a better estimate for how an ambient light image would have been had it been captured at the time that fluorescence light frame 2 510 was captured. However, ambient light frame 1 506 could be used, such as by scaling the estimate of optical flow according to the ratio of (T-t)/T.

Returning to method 400 depicted in FIG. 4, at step 408, a combined image is generated based on at least the estimated first light source image generated at step 406 and the second light source image 462. The combined image may be, for example, an ambient light compensated fluorescence image generated based on a fluorescence light image and an estimate of an ambient light image corresponding with the capture time of the fluorescence light image. FIG. 5 illustrates this example. The ambient light frame estimate 514 is combined with the fluorescence light frame 2 510 to generate an ambient light compensated fluorescence frame 2 516. This step may be similar to step 310 of method 300 in which at least a proportion of the ambient light frame estimate 514 (for example, the ambient light frame estimate may be scaled according to an effective gain and/or according to a level of ambient light) is subtracted from fluorescence light frame 2 510.

The combined image may be an image generated using an image generated from the estimated first light source image and the second light source image in combination with another image. For example, with reference to FIG. 5, the ambient light compensated florescence frame 2 516 may be combined with reflected light frame 2 508 to generate a combined reflected light and fluorescence frame 520. Given that the ambient light compensated fluorescence frame 2 516 corresponds to a different capture time than the reflected light frame 2 508, the estimate of optical flow 550 may be used to generate an estimate of the ambient light compensated fluorescence frame 2 516 for the capture time of reflected light frame 2 508 using the scaling approach described above (e.g., scaling the optical flow estimate by a ratio of the difference in capture time between reflected light frame 2 508 and fluorescence light frame 2 510 and the difference in capture time between the ambient light frames used to generate the estimate of optical flow 550), resulting in the ambient light compensated fluorescence frame estimate 518 depicted in FIG. 5. This estimated ambient light compensated image can then be combined with the reflected light image to produce a combined reflected light and fluorescence image without (or with minimized) motion artifacts to generate the combined reflected light and fluorescence frame 520.

Method 400 may include the optional step 410 of displaying the combined image generated in step 408. The combined image may be displayed to medical personnel during a medical procedure, for example, as a frame of a video stream. Additionally or alternatively, the combined image may be analyzed to determine one or more characteristics of tissue captured in the image as discussed above with respect to step 312 of method 300.

As noted above, the estimate of optical flow can be generated from any of the light source series. For example, the estimate of optical flow may be generated from the first light source series 450, the second light source series 452, the third light source series 454, or any other light source series in the time series of images. In the example of FIG. 5, the estimate of optical flow was generated from the same light source series (the first light source series 450 for the example of FIG. 5) from which the estimated image (the estimated first light source series for the example of FIG. 5) was generated. In other words, the optical flow estimate was generated from the ambient light frames to generate the ambient light frame estimate. However, the estimate of optical flow could have been generated from the fluorescence frames (the second light source series 452 for the example of FIG. 5) or from the reflected light frames (the third light source series 454 for the example of FIG. 5).

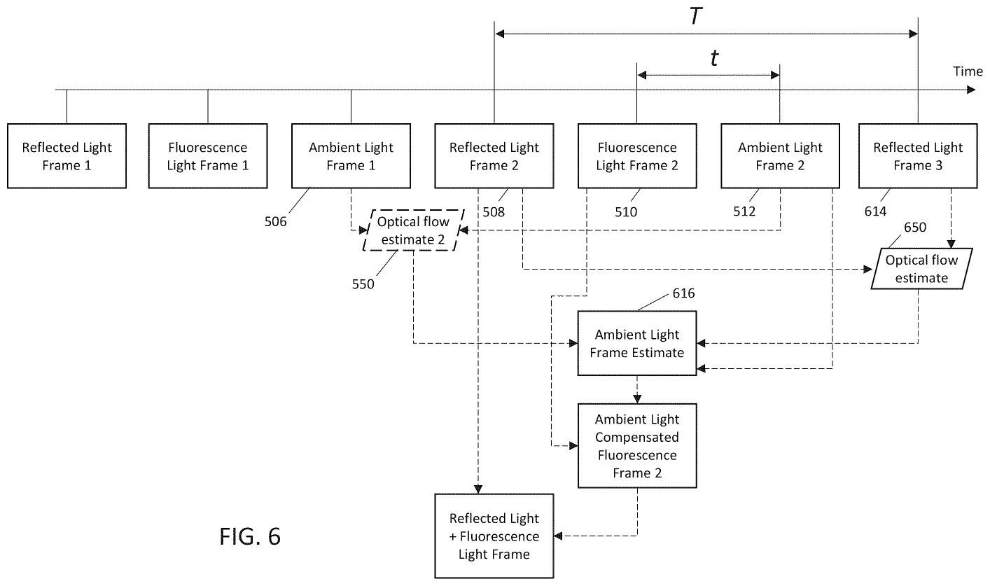

FIG. 6 illustrates an example similar to FIG. 5 but where the estimate of optical flow is generated from the reflected light frames. Relative to FIG. 5, the series of frames in FIG. 6 is expanded by one reflected light frame (reflected light frame 3 614). In this example, the optical flow estimate (optical flow estimate 650) is generated from reflected light frames-reflected light frame 2 508 and reflected light frame 3 614. Like in the example of FIG. 5, the estimate of optical flow 550 of FIG. 6 is used to shift ambient light frame 2 512 to correspond to the capture time of fluorescence light frame 2 510. However, the optical flow estimate of FIG. 6 is scaled differently than in the example of FIG. 5—it is scaled according to the ratio of the difference between the capture times of fluorescence light frame 2 510 and ambient light frame 2 512 and the difference between the capture times of the reflected light frames used to generate the optical flow estimate (reflected light frame 2 508 and reflected light frame 3 614). Using the reflected light frames instead of the ambient light frames to generate the estimate of optical flow may be preferable since the relatively higher intensity of reflected light images relative to the ambient light images may lead to a more accurate estimate, in particular if the ambient light image intensity is low.

As noted above, multiple optical flow estimates generated from different light source series may be used to generate the estimated image. For example, as depicted in FIG. 6, the estimate of optical flow 550 from the reflected light frames 508 and 614 and the estimate of optical flow 660 from the ambient light frames 506 and 512 may be used to generate the ambient light frame estimate 616. The two estimates of optical flow 550 and 660 could be applied as a weighted sum, which could weight the estimates of optical flow equally or weight one estimate of optical flow more favorably than another-such as weighting the reflected light frame-based estimate of optical flow 550 more favorably given that its relatively higher intensities may provide a better estimate of the motion-based shift that should be applied to the ambient light frame 512.

As depicted in FIG. 6, the ambient light frame estimate 616 may be used in combination with fluorescence light frame 2 510 to generate an ambient light compensated fluorescence frame 2. The ambient light compensated fluorescence frame 2 may be combined with reflected light frame 2 508 to generate a combined reflected light and fluorescence light frame.

FIG. 5 and FIG. 6 illustrate examples of estimating ambient light images to generate ambient light compensated fluorescence images that, for example, may be displayed or combined with reflected light images. However, method 400 need not be limited to ambient light compensation. Method 400 could be used to combine any different light source images that are captured at different times.

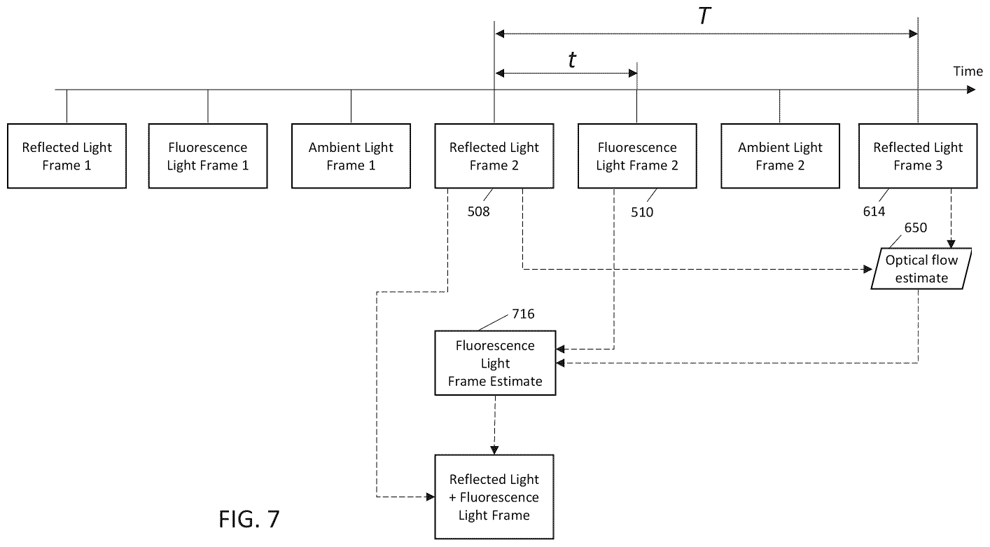

FIG. 7 illustrates an example of using method 400 to combine fluorescence images and reflected light images. The series of frames shown in FIG. 7 is the same as the series of frames shown in FIG. 6. However, the series need not include the ambient light frames. In the example of FIG. 7, the optical flow estimate 650 is generated from reflected light frames (reflected light frames 2 508 and 3 614). The optical flow estimate is used to shift fluorescence light frame 2 510 back in time to the capture time of reflected light frame 2 508 to generate a fluorescence light frame estimate 716. Similar to the examples of FIG. 5 and FIG. 6, the optical flow estimate 650 can be scaled according to the difference (duration T) in the capture times of the reflected light images used to generate the optical flow estimate and the difference (duration 1) in the capture times of the frame from which the estimated frame is generated (fluorescence light frame 2 510) and the capture time of the frame for which the estimated frame is generated. As depicted in FIG. 7, the fluorescence light frame estimate 716 can be combined with the reflected light frame 2 to generate a combined reflected light and fluorescence light frame.

As noted above, method 400 and method 300 can be used in combination. For example, an estimated ambient light image generated according to method 400 (a motion compensated ambient light image) can be used to compensate for ambient light in a fluorescence image when the level of ambient light is above a threshold according to method 300. As such, step 310 of method 300 may include steps 404 and 406 of method 400. The estimated ambient light image can be scaled according to an effective gain scaling factor as discussed above with respect to step 310 and can be scaled according to an ambient light scaling factor if the level of ambient light determined at step 304 is between lower and upper thresholds, as discussed above for method 300.

Although the description of methods 400-700 of FIGS. 4-7 above often refers to the order of frame capture depicted in the exemplary timing scheme of FIG. 2A, it will be readily understood by a person of skill in the art that the methods are not tied to this particular order. Rather, any order of the frames may be used. For example, methods 400-700 of FIGS. 4-7 may be practiced with the timing scheme of FIG. 2B.

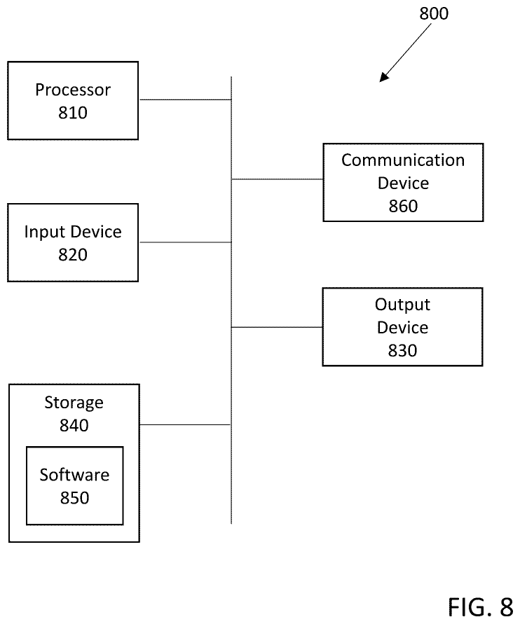

FIG. 8 illustrates an example of a computing system 800 that can be used for one or more components of system 100 of FIG. 1, such as one or more of light source 108, camera control unit 120, image acquisition assembly 104, and image processing unit 122. System 800 can be a computer connected to a network, such as one or more networks of hospital, including a local area network within a room of a medical facility and a network linking different portions of the medical facility. System 800 can be a client or a server. System 800 can be any suitable type of processor-based system, such as a personal computer, workstation, server, handheld computing device (portable electronic device) such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices as described above), such as programming for performing one or more steps of method 300 of FIG. 3 and/or method 400 of FIG. 4.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer readable medium can include, but is not limited to, an electronic, magnetic, optical, electromagnetic, or infrared wired or wireless propagation medium.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for compensating for ambient light in medical imaging, the method comprising:
receiving an ambient light image and a fluorescence image;
determining a level of ambient light in the ambient light image; and
in accordance with determining that the level of ambient light in the ambient light image meets a threshold, generating an ambient light compensated image based on the ambient light image and the fluorescence image that compensates for contributions of the ambient light to the fluorescence image.

2. The method of claim 1, wherein determining the level of ambient light in the ambient light image comprises determining a proportion of the ambient light image that has pixel intensity values that are above a predetermined amount.

3. The method of claim 1, wherein the threshold corresponds to a proportion of an image having pixel values above a predetermined amount.

4. The method of claim 1, wherein the ambient light compensated image comprises the fluorescence image modified based on the ambient light image.

5. The method of claim 1, wherein the ambient light compensated image comprises a combination of a reflected light image and the fluorescence image modified to compensate for the contributions of the ambient light to the fluorescence image.

6. The method of claim 5, wherein generating the ambient light compensated image comprises:
generating a compensated fluorescence image by subtracting at least a portion of the ambient light image from the fluorescence image; and
combining the compensated fluorescence image with the reflected light image.

7. The method of claim 1, wherein generating the compensated fluorescence image comprises scaling pixel values of at least one of the ambient light image and the fluorescence image based on at least one of a difference in exposure period and a difference in gain.

8. The method of claim 1, wherein combining the compensated fluorescence image with the reflected light image comprises scaling pixel values of at least one of the compensated fluorescence image and the reflected light image based on at least one of a difference in exposure period and a difference in gain.

9. The method of claim 1, further comprising, in accordance with determining that the level of ambient light in the ambient light image does not meet the threshold, displaying the fluorescence image or an image generated based on the fluorescence image without compensating for the ambient light.

10. The method of claim 1, wherein generating the ambient light compensated image comprises scaling pixel values of the ambient light image by a scaling factor that corresponds to an amount that the level of ambient light in the ambient light image is above the threshold.

11. The method of claim 1, wherein the threshold is a lower threshold, and generating the ambient light compensated image comprises:
scaling pixel values of the ambient light image by a scaling factor when the level of ambient light in the ambient light image is above the lower threshold and below an upper threshold; and
not scaling the pixel values of the ambient light image by the scaling factor when the level of ambient light in the ambient light image is above the upper threshold.

12. The method of claim 1, wherein the ambient light compensated image is generated based on an estimated ambient light image that is an estimate of the ambient light at a capture time of the fluorescence image.

13. The method of claim 1, comprising displaying the ambient light compensated image.

14. The method of claim 1, comprising generating and displaying a visual guidance based on the ambient light compensated image.

15. A system comprising one or more processors, memory, and one or more programs stored in the memory for execution by the one or more programs, the one or more programs including instructions for:
receiving an ambient light image and a fluorescence image;
determining a level of ambient light in the ambient light image;
in accordance with determining that the level of ambient light in the ambient light image meets a threshold, generating an ambient light compensated image based on the ambient light image and the fluorescence image that compensates for contributions of the ambient light to the fluorescence image; and
displaying the ambient light compensated image.

16. A method for combining images captured at different times, the method comprising:
receiving a time series of images that comprises multiple series of different light source images, the multiple series of different light source images comprising a first light source series and a second light source series, wherein the first light source series comprises a first light source image and the second light source series comprises a second light source image that was captured at a different time than the first light source image;
generating at least one estimate of optical flow based on at least one of the multiple series of different light source images;
generating, based on the first light source image and the at least one estimate of optical flow, an estimated first light source image that is an estimate of a first light source image captured at a capture time of the second light source image; and
generating a combined image based on at least the estimated first light source image and the second light source image.

17. The method of claim 16, wherein the first light source image is an ambient light image, the second light source image is a fluorescence image, and the estimated first light source image is an estimated ambient light image.

18. The method of claim 17, wherein the combined image is an ambient light compensated fluorescence image or a combination of a reflected light image with the ambient light compensated fluorescence image.

19. The method of claim 17, comprising, prior to generating the estimated ambient light image, determining a level of ambient light in the ambient light image, comparing the level of ambient light in the ambient light image to a threshold, and generating the estimated ambient light image in accordance with the level of ambient light in the ambient light image meeting the threshold.

20. The method of claim 16, wherein the first light source image is a fluorescence image, the second light source image is a reflected light image, and the estimated first light source image is an estimated fluorescence image.

21. The method of claim 16, wherein generating the estimated first light source image comprises spatially shifting pixel values of the first light source image based on the at least one estimate of optical flow.

22. The method of claim 16, wherein generating the combined image comprises subtracting at least a proportion of the estimated first light source image from the second light source image.

23. The method of claim 22, wherein the time series of images comprises a third light source image, and generating the combined image comprises combining the third light source image with a result of the subtraction of the estimated first light source image from the second light source image.

24. The method of claim 16, wherein a capture time of the first light source image is between capture times of images of at least one of the multiple series of different light source images used to generate the at least one estimate of optical flow.

25. The method of claim 24, wherein the at least one estimate of optical flow is generated based on the second light source image and another image of the second light source series and a capture time of the first light source image is closer in time to a capture time of the second light source image than a capture time of the other image of the second light source series.

26. The method of claim 24, wherein generating the estimated first light source image comprises spatially shifting pixel values of the first light source image based on at least one optical flow vector of the at least one estimate of optical flow that is scaled based on a ratio between: (1) a time between the capture time of the second light source image and the capture time of the first light source image, and (2) a time between the images of at least one of the multiple series of different light source images used to generate the at least one estimate of optical flow.

27. The method of claim 24, wherein the time series of images comprises a third light source series, and the at least one estimate of optical flow comprises a first estimate of optical flow generated based on the third light source series and a second estimate of optical flow generated based on the second light source series.

28. The method of claim 27, wherein generating the estimated first light source image comprises spatially shifting pixel values of the first light source image based on a weighted sum of the first and second estimates of optical flow.

29. The method of claim 16, wherein the time series of images comprises a third light source series, and the at least one estimate of optical flow is generated based on the third light source series.

30. The method of claim 29, wherein a capture time of the first light source image is between capture times of images of the third light source series used to generate the at least one estimate of optical flow.

31. The method of claim 16, wherein the at least one estimate of optical flow comprises multiple flow vectors corresponding to different regions of the multiple images of the time series of images.

32. The method of claim 31, wherein generating the estimated first light source image comprises spatially shifting pixel values of different regions of the first light source image based on corresponding flow vectors of the multiple flow vectors.

33. The method of claim 16, wherein the at least one estimate of optical flow comprises a single flow vector, and generating the estimated first light source image comprises spatially shifting the first light source image based on the single flow vector.

34. The method of claim 16, wherein the time series of images comprises open-field images and the first light source image comprises room light.

35. The method of claim 16, wherein the time series of images comprises endoscopic images and the first light source image comprises light from a light source that is not for capturing the time series of images.

36. The method of claim 16, comprising displaying the combined image.

37. A system comprising one or more processors, memory, and one or more programs stored in the memory for execution by the one or more programs, the one or more programs including instructions for:
receiving a time series of images that comprises multiple series of different light source images, the multiple series of different light source images comprising a first light source series and a second light source series, wherein the first light source series comprises a first light source image and the second light source series comprises a second light source image that was captured at a different time than the first light source image;
generating at least one estimate of optical flow based on at least one of the multiple series of different light source images;
generating, based on the first light source image and the at least one estimate of optical flow, an estimated first light source image that is an estimate of a first light source image captured at a capture time of the second light source image; and
generating a combined image based on at least the estimated first light source image and the second light source image.
